(19) United States
(12) Patent Application Publication
(10) Pub. No.: US 2006/0008542 A1
(43) Pub. Date: Jan. 12, 2006

(54) Preparation containing a mixture of picrorrhiza and ginkgo for the relief of disease

(76) Inventor: Nicholas John Larkins, London (GB)

Correspondence Address: FOLEY AND LARDNER LLP;SUITE 500, 3000 K STREET NW, WASHINGTON, DC, 20007, US

(21) Appl. No.: 10/512,202

(22) PCT Filed: Apr. 16, 2003
(86) PCT No.: PCT/GB03/01644

§ 371 Date: Aug. 22, 2005

(30) Foreign Application Priority Data
Apr. 23, 2002 (GB) 0209254.2

(51) Int. Cl. A61K 36/16

(52) U.S. Cl. 424/752

(57) ABSTRACT

Improved pharmaceutical compositions which include Ginkgo biloba and Picrorrhiza kurroa or extracts thereof, having a reduced amount of (or which do not include) components of natural Ginkgo biloba and natural Picrorrhiza kurroa which have effects which are detrimental to the effect or the pharmaceutical.

The present invention relates to preparations for the treatment or relief of diseases, especially inflammatory diseases, in humans and animals. In particular it relates to improved preparations for the treatment of inflammatory respiratory diseases such as asthma in humans, especially in children.

Asthma is a chronic disease of the airways characterised by recurrent airway obstruction. Known treatments of asthma include daily doses of pharmaceuticals such as inhaled corticosteroids, antihistamines and inhaled beta agonist bronchodilators such as Ventolin (RTM). The aim of such treatments is to manage or ameliorate the condition, that is to provide symptomatic relief of wheezing and/or breathlessness and/or coughing. Unfortunately, even with such treatments, asthma sufferers are prone to severe asthma attacks or crises which may be brought on or exacerbated by other illness such as colds or influenza, allergens (for example pollen, house dust etc.) or by exercise. Such crisis points may involve unremitting coughing and other respiratory distress, and may necessitate hospitalisation and/or out patient emergency care to administer repeated bronchodilation therapy, oxygen therapy, and/or high doses of oral glucocorticosteroids.

A major goal of asthma therapy is the prevention of crises which require emergency therapy and or/oral corticosteroid treatment. This is for two major reasons. First, it is the crises which are the major cause of the morbidity and mortality associated with asthma. Secondly, recurrent steroid use, especially at high doses of oral steroids, is associated with multiple adverse affects, which are well documented.

Inhalation therapy of asthma using an inhaler is the standard treatment for attacks other than severe acute attacks, but has several disadvantages. It requires co-ordination of discharge of the inhaler with inhalation, which many people, especially children, find difficult. It also requires considerable respiratory effort which those suffering from asthma find difficult.

Beta 2 bronchodilators, e.g. salbutamol (Ventolin [RTM]) and terbutaline (Bricanyl [RTM]) are usually taken by inhalation, but may be taken orally. When taken orally, these compounds have considerable adverse effects, such as inducing tremor and/or cardiovascular side effects (such adverse effects can also follow inhalation administration but are less likely). The side effects are particularly bad at high doses. Oral steroids e.g. prednisolone and prednisone, which are used in acute asthma attacks, also have known (and considerable) side effects; these compounds are generally taken for a short period in order to alleviate the attack while avoiding those side effects.

Thus, known oral treatments for asthma either suffer from the severe and well known side effects associated with glucocorticosteroids, or have not proved very effective. There is therefore a considerable need for an effective oral treatment for asthma and other respiratory diseases.

The present inventor has previously developed compositions for the treatment of disease which include natural Ginkgo-biloba and natural Picrorrhiza kurroa and, optionally, apocynin.

Ginkgo biloba and Picrorrhiza kurroa are both plants. As such, they are extremely complicated mixtures of separate components. The present inventor has found that a number of components in natural Ginkgo biloba and natural Picrorrhiza kurroa have pro-inflammatory effectiveness (that is, they may cause inflamation); these components are thus detrimental to the overall utility or effectiveness of the composition. The present invention provides improved compositions which have a reduced amount of (or do not include) these components, and methods by which these compositions may be made.

According to the present invention there is provided a pharmaceutical composition or food supplement comprising:
a mixture of Picrorrhiza kurroa and Ginkgo biloba which includes less of a component of natural Picrorrhiza kurroa than the amount of that component present in natural Picrorrhiza kurroa or less of a component of natural Ginkgo biloba than the amount of that component present in natural Ginkgo biloba;
which component (hereafter referred to as “Component”) if isolated e.g. as a single species, gives a substantially negative result in a neutrophil oxidative burst assay (for example, a negative value, e.g. a value more negative than −10%, in the test set out in section 3 below); provided that the Picrorrhiza kurroa, which includes less of a Component of natural Picrorrhiza kurroa than the amount of that Component present in natural Picrorrhiza kurroa, does not contain apocynin or apocynin derivative as sole pharmacologically active species.

Preferred pharmaceutical compositions or food supplements comprise a mixture of Picrorrhiza kurroa and-Ginkgo biloba which includes less of a first Component of natural Picrorrhiza kurroa than the amount of that first Component present in natural Picrorrhiza kurroa and less of a second Component of natural Ginkgo biloba than the amount of that second Component present in natural Ginkgo biloba.

Preferably, the mixture includes less of a plurality of Components of natural Picrorrhiza kurroa than the amount of those Components present in natural Picrorrhiza kurroa and/or less of a plurality of Components of natural Ginkgo biloba than the amount of those Components present in natural Ginkgo biloba.

Preferably the composition includes less of a Component of natural Picrorrhiza kurroa than the amount of that Component present in natural Picrorrhiza kurroa.

In this specification the terms “natural Ginkgo biloba”, “natural Picrorrhiza kurroa” and “natural product” refer to an unresolved mixture of compounds in the form of an unpurified plant or root or a commercially available product. The terms “Ginkgo biloba” and “Picrorrhiza kurroa” refer to a mixture of components obtained or obtainable from the natural product (e.g. by extraction); they are not intended to mean the natural product, or a single species which is present in the natural product but which is isolated from, isolable from, or otherwise obtainable from the natural product (such as apocynin which is a single species present in natural Picrorrhiza kurroa). Thus, certain mixtures of compounds (referred to by Extract No. below) which, if used in pharmaceutical compositions or food supplements give rise to the therapeutic activity and/or other beneficial effects, may be classed as “Ginkgo biloba” and “Picrorrhiza kurroa”.

By “apocynin or apocynin derivative” is meant the plant-phenol 4-hydroxy-3-methoxyacetophenone, tautomers thereof, and derivatives thereof, such as dimeric forms of apocynin, in particular the symmetrical dimer formed through the formation of a 5, 5′ C—C bond, and natural derivatives such as acetoguiacone and androsin (a glycoside derivative of apocynin).

Preferably the pharmaceutical composition or food supplement contains the Component in an amount which is less than 70% w/w, more preferably less than 50% w/w, even more preferably less than 10% w/w, of the amount present in natural Picrorrhiza kurroa and/or natural Ginkgo biloba. Desirably, the pharmaceutical composition contains the Component in an amount which is substantially zero. The Components which give a substantially negative result in a neutrophil oxidative burst assay may include certain sugars, fatty acids and polyunsaturated fatty acids, and/or the specific Components as discussed below.

Preferably the pharmaceutical composition or food supplement includes less of a Component of natural Picrorrhiza kurroa than the amount of that Component present in natural Picrorrhiza kurroa, wherein the Component is among the most polar component(s) of natural Picrorrhiza kurroa. By most polar is meant a component (or Component) having log P of −2 or more negative, where P is the Partition Ratio between Octanol/Water. Examples of such most polar Components are catalpol, verminoside and aucubin.

Preferably the pharmaceutical composition or food supplement includes less of a Component of natural Picrorrhiza kurroa than the amount of that Component present in natural Picrorrhiza kurroa, wherein the Component is among the least polar component(s) of natural Picrorrhiza kurroa. By least polar is meant a component (or Component) having log P of 5 or greater. Examples of such Components include tripalmitin.

Preferably the pharmaceutical composition or food supplement includes less of a Component of natural Ginkgo biloba than the amount of that Component present in natural Ginkgo biloba, wherein the Component is among the least polar component(s) of natural Ginkgo biloba. Examples of such Components include 10-heptacosanol, 10-nonacosanol, 13-nonacosanol, amentoflavone, bilobetin, ginkgetin, ginkgolic-acid, isoginkgetin, nonacosane, octacosanol-1, sciadopitysin, spinasterol and stigmasterol, and/or derivatives, plant metabolites or residues of any of these.

According to the present invention in a further aspect, a pharmaceutical composition or food supplement comprises:
a mixture of Picrorrhiza kurroa and Ginkgo biloba which includes less of a Component of natural Picrorrhiza kurroa than the amount of that Component present in natural Picrorrhiza kurroa;
which Component has log P of less than about −2 or of greater than about +5 provided that the Picrorrhiza kurroa, which includes less of a Component of natural Picrorrhiza kurroa than the amount of that Component present in natural Picrorrhiza kurroa, does not contain apocynin or apocynin derivative as sole pharmacologically active species.

According to the present invention there is provided a pharmaceutical composition or food supplement comprising:
a mixture of Picrorrhiza kurroa and Ginkgo biloba which includes less of a component of natural Picrorrhiza kurroa than the amount of that component present in natural Picrorrhiza kurroa;
which component, hereafter referred to as “Komponent”, is a cucurbitacin; provided that the Picrorrhiza kurroa, which includes less of a Komponent of natural Picrorrhiza kurroa than the amount of that Komponent present in natural Picrorrhiza kurroa, does not contain apocynin or apocynin derivative as sole pharmacologically active species.

By “cucurbaticin” is meant a single cucurbitacin (e.g. any one of the cucurcbaticins mentioned in the table immediately below) or a corresponding cucurbitacin glycoside; “cucurbaticins” refers to unresolved mixtures of one or more cucurbitacins and/or cucurbitacin glycocide compounds. The cucurbitacins are not pro-inflammatory (i.e. they are not Components as discussed above), but they may give rise to side effects (e.g. upset stomach at high doses) and they have been shown to be cytotoxic, and it is thus desirable that the mixtures/pharmaceutical compositions and/or food supplements include as little of these as possible. The Table immediately below lists the cucurbitacins present in natural Picrorrhiza kurroa together with their log P value. The cucurbitacins may be removed by conventional separation techniques (e.g the sovent extraction techniques discussed below and in section 3) and the skilled man will appreciate and readily devise techniques suitable to remove the cucurbitacin(s)from the natural Picrorrhiza kurroa.

TABLE

| | | |
|---|---|---|
| cucurbitacin B | log P | 2.5 ± 0.6 |
| cucurbitacin D | log P | 1.7 ± 0.6 |
| cucurbitacin E | log P | 3.4 ± 0.6 |
| cucurbitacin F | log P | 1.9 ± 0.6 |
| cucurbitacin I | log P | 2.5 ± 0.5 |
| cucurbitacin R | log P | 1.9 ± 0.6 |

Thus, more preferred pharmaceutical compositions and food supplements are those which contain at least one cucurbitacin in an amount which is less than 10% w/w of the amount present in natural Picrorrhiza kurroa. The amount of cucurbitacins is preferably less than 3 mg, more preferably less than 0.1 mg, per 100 mg of Picrorrhiza kurroa in the mixture.

Desirably, the pharmaceutical composition or food supplement is one in which the physiologically active proportion of cucurbitacins is substantially zero.

Preferably the pharmaceutical compositions and food supplements include a lesser proportion of inactive components than are found in natural Ginkgo biloba and/or natural Picrorrhiza kurroa. The inactive components simply reduce the potency of the product. Examples of inactive components are calcium oxalate leaf (from natural Ginkgo biloba) and D-Mannitol (from natural Picrorrhiza kurroa).

Preferred pharmaceutical compositions and food supplements give a positive Assay score of 10% or greater, preferably 50% or greater, in the neutrophil oxidative burst assay set out in section 3 herein.

Preferably the pharmaceutical composition or food supplement is substantially similar to, e.g. in the assay set out in section 3 gives a score of between +5% and −5% of the value of, that obtained or obtainable by a method comprising a step of extraction from the natural Ginkgo biloba or natural Picrorrhiza kurroa. Preferably the extraction is extraction using polar organic solvent. Preferably the polar organic solvent is an alkanol, preferably a lower (C1 to C6) alkanol. Preferred solvents include methanol or ethanol. Suitable methods are well known in the art (for example Soxhlet extraction).

Preferably the pharmaceutical composition or food supplement is obtained or obtainable by a method comprising a step of fractionation, preferably a chromatography step. It will be appreciated that herein “fractionation”, “chromatography” etc. may inherently include one or more steps of, for example, separation, identification and removal of the relevant product of the fractionation, e.g. chromatography step. Preferably the chromatography is by reverse phase chromatography (e.g. HP20 chromatography). The chromatography may also be normal phase chromatography, for example using alumina or silica. Preferably the normal phase chromatography uses a non polar organic solvent (eg hexane), a slightly polar organic solvent (eg ethyl acetate) followed by a polar organic solvent (e.g. methanol). The treatment may include reverse phase chromatography and/or normal phase chromatography.

It is preferred that the treatment includes an extraction step and a chromatography step, preferably an extraction step followed by a chromatography step.

Preferred pharmaceutical compositions or food supplements are obtained or obtainable by a method comprising the steps of:

a) reverse phase chromatography of natural Picrorrhiza kurroa; and

b) normal phase chromatography of the product of step (a) to remove (or lower the proportion of) Component(s) and/or Komponent(s) of natural Picrorrhiza kurroa (preferably by a process which generates a Extract substantially similar to Extract 10 and/or Extracts 14, 16, 17, 18, 30, 32 or 33, discussed below);

or equivalent steps producing a substantially similar product.

Preferably, the method may further comprise a step of extraction (e.g. solvent extraction) from Picrorrhiza kurroa. The extraction may, for example, be from natural Picrorrhiza kurroa itself (e.g. from the leaves) or from a commercially available natural Picrorrhiza kurroa product.

Preferably, step a) uses a polymeric resin (e.g. HP20) solid phase. Preferably step b) uses a silica solid phase. Preferably step b) is a step of fractionation by flash chromatography on silica.

Step b) may remove substantially all of the least and most polar component(s). Preferably, products of step (b) include a Extract substantially similar to Extract 10 and/or any one of Extracts 14, 16, 17, 18, 30, 32 or 33 discussed below. Preferably, step (b) generates a product which contains at least 50%, preferably at least 80% (both quantitatively and qualitatively) of the same components as Extract 10 and/or any one of Extracts, 14, 16, 17, 18, 30, 32 or 33.

Preferred pharmaceutical compositions or food supplements are obtained or obtainable by a method comprising the steps of:

c) reverse phase chromatography of natural Ginkgo biloba; and

d) normal phase chromatography of the product of step (d) to remove (or lower the proportion of) Component(s) of natural Ginkgo biloba (preferably by a process which generates a Extract substantially similar to one or more of Extract 41, 44, 50, 54, 55, 59, 60, 61 and 64, discussed below);

or equivalent steps producing a substantially similar product.

Preferably, the method may further comprise a step of extraction (e.g. solvent extraction) from Ginkgo biloba. The extraction may, for example, be from natural Ginkgo biloba itself (e.g. from the leaves) or from a commercially available natural Ginkgo biloba product.

Preferably, step c) uses a polymeric resin (e.g. HP20) solid phase. Preferably step d) uses a silica solid phase. Preferably step d) is a step of fractionation by flash chromatography on silica.

Step d) may remove substantially all of the least and most polar component(s). Preferably, products of step (d) include a Extract substantially similar to Extract 54 and/or any one of Extracts 41, 44, 50, 55, 59, 60, 61 and 64 discussed below. Preferably, step (d) generates a product which contains at least 50%, preferably at least 80% (both quantitatively and qualitatively) of the same components as Extract 54 and/or any one of Extracts 41, 44, 50, 55, 59, 60, 61 and 64.

Preferably the pharmaceutical composition is a preparation for human or veterinary use. We also provide the use as food supplements for humans and/or animals.

The presence of fewer and/or a lower proportion of Component(s) and/or Komponent(s) (compared to a mixture of natural Ginkgo biloba and natural Picrorrhiza kurroa) enhances the overall anti-inflammatory potency and/or reduces side effects of the pharmaceutical compositions/preparations of the invention. It will be appreciated that compositions wherein only a portion of either the natural Ginkgo biloba or the natural Picrorrhiza kurroa has been treated to remove (or lower the proportion of) the Components and/or Komponent(s) are within the scope of the invention(s). However, it is preferred that substantially all of the natural Ginkgo biloba and especially the natural Picrorrhiza kurroa are so treated.

The present inventor has recognised and isolated the “active” components in natural Picrorrhiza kurroa and natural Ginkgo biloba which have anti-inflammatory potency, and, surprisingly, shown that there are beneficial, e.g. synergistic, effects shown by specific combinations of two or more of these active components.

According to the present invention there is provided a pharmaceutical composition or food supplement comprising a mixture of an Extract of natural Ginkgo biloba and an Extract of natural Picrorrhiza kurroa, each of which Extracts if isolated e.g. as a single species, gives a substantially positive result in a neutrophil oxidative burst assay (for example, a value more positive than +10, in the test set out in section 3 below); provided that the Extract of natural Picrorrhiza kurroa does not contain apocynin or apocynin derivative as the sole pharmacologically active species.

The term Extract refers to a component or mixture of components obtained or obtainable by extraction from the natural product; it is not intended to mean the natural product. The term Extract includes a dried Extract (for example in the form of a solid, for example a powder or gum).

Preferably the Extract is such as is obtained or obtainable by a method which comprises a step of extraction from the natural Ginkgo biloba or natural Picrorrhiza kurroa. Preferably the extraction is extraction using a polar organic solvent, preferably a polar organic solvent such as an alkanol, preferably a lower (C1 to C6) alkanol. Preferred solvents include methanol or ethanol.

Preferably, the Extract is substantially similar to (gives an assay score in the test set out in sections 3 between about +5% and about −5% of the value given by) that obtained or obtainable by a method which comprises a step of fractionation, preferably a chromatography step. Preferably the chromatography is by reverse phase chromatography (e.g. HP20 chromatography). The chromatography may also be normal phase chromatography, for example using silica. The Extract may be such as is obtained or obtainable by a method that may include reverse phase chromatography and/or normal phase chromatography.

Preferably the pharmaceutical composition or food supplement comprises an Extract of natural Ginkgo biloba which has log P of between −1.9 and 4.9, more preferably between 1 and 2.5. Also preferred are Extracts which have log P of between −1.9 and 4.9 but which metabolise (e.g. in the gut) to components which have log P between 1 and 2.5. Preferred Extracts of natural gingko biloba may include quercetin, quercetin-3-0-rutinoside and/or quercetin-3-rhamnoglucoside. Preferred Extracts of natural Ginkgo biloba may also include one or more of 6-hydroxykynurenic-acid, acacetin, apigenin, bilobalide, d-catechin, d-gallocatechin, isorhamnetin, kaempferol, 1-epicatechin, 1-epigallocatechin, luteolin, sequoyitol, shikimic-acid, and tricetin and pharmaceutically active derivatives, plant metabolites or residues of any of these.

Preferably the pharmaceutical composition or food supplement comprises an Extract of natural Picrorrhiza kurroa which has log P of between −1.9 and 4.9, more preferably between 1 and 2.5. Also preferred are Extracts of natural Picrorrhiza kurroa which have log P of between −1.9 and 4.9 but which metabolise (e.g. in the gut) to components which have log P between 1 and 2.5. An Example of the latter Extract is androsin (log P −1.2) which undergoes glucosidic cleavage to yield apocynin (log P of 1.6).

Preferred Extracts of natural Picrorrhiza kurroa include apocynin, androsin and the picrosides, for example one or more of picroside I, III and IV. Preferred such Extracts of natural Picrorrhiza kurroa may also include one or more of arvenin-III, PICRORHIZIN, veronicoside, picroside II, kutkoside, pikuroside, scroside A scroside B, scroside C, plantamajoside and picein and pharmaceutically active derivatives, plant metabolites or residues of any of these.

Preferred pharmaceutical compositions or food supplements are those having an Assay score of 60% in the assay set out in section 3 herein.

The applicant's pharmaceutical compositions and food supplements are advantageous because they enable administration of components of natural Ginkgo biloba and natural Picrorrhiza kurroa which have anti-inflammatory activity without having to administer the plants (natural products) themselves. In other words, to increase the dose of the anti-inflammatory species/Extract one can, using the applicants compositions, simply administer more of that species/Extract; it is no longer necessary to increase the proportion of active (e.g. anti-inflammatory component) by increasing the dose of natural Ginkgo biloba and/or natural Picrorrhiza kurroa (which might lead to side effects, such as diarrhoea).

The Extracts in the specification shall be referred to by their “Extract Numbers”, indicated in the description of their preparation (described in sections 1 to 5 below). It should be noted that the Extract Numbers do not necessarily refer to absolutely pure components; they may be mixtures of components.

Preferred Extracts include Extracts 10, 14, 16, 17, 18, 30, 31, 32 and 33 (Extracts of natural Picrorrhiza *kurroa) and Extracts 41, 44, 50, 54, 55, 59, 60, 61 and 64 (Extracts of natural Ginkgo biloba), and extracts which are substantially similar to any thereof.

Preferred Extracts may include fatty acids, iridoid glycosides (e.g., Extract 31), some flavonoids or flavonoid glycosides (e.g. Extract 41) quercitrin (e.g., Extract 54) and campherol (e.g., Extract 64). Extracts may include ginkgolides.

Preferably the pharmaceutical composition or food supplement includes an Extract of natural Ginkgo biloba and an Extract of natural Picrorrhiza kurroa which represent a synergistic combination of Extracts, e.g. a combination of Extracts which gives a more positive result in the neutrophil oxidative burst assay mentioned in section 3 than would be expected from the simple additive value of the assay scores of the individual Extracts. Preferred such combinations include Extracts formed by the combination of the following pairs of Extracts: Extract Numbers 17 and 59; 10 and 41; 17 and 50; 17 and 41; and 17 and 54 discussed below; and combinations or compositions substantially similar to these combinations.

Preferred pharmaceutical compositions and food supplements according to the invention include two (or more) Extracts of natural Ginkgo biloba, each of which Extracts, if isolated e.g. as a single species, gives a substantially positive result in a neutrophil oxidative burst assay.

Preferred pharmaceutical compositions and food supplements are those which include combinations of two (or more) Extracts of natural Ginkgo biloba which represent a synergistic combination of Extracts. Preferred such combinations include Extracts formed by the combination of the following pairs/trios of Extracts: Extract Numbers 50, 54 and 59; 54 and 59; 54 and 64; 42 and 44; 50, 59 and 64; 42, 43 and 44; 50 and 59; 54, 59 and 64; 50. and 54; 50 and 64; 43 and 44; and 50, 54 and 64, and combinations of Extracts or compositions substantially similar to any of these.

Preferred pharmaceutical compositions and food supplements according to the invention include two (or more) Extracts of natural Picrorrhiza kurroa, each of which Extracts, if isolated e.g. as a single species, gives a substantially positive result in a neutrophil oxidative burst assay.

Preferred pharmaceutical compositions and food supplements are those which include combinations of two (or more) Extracts of natural Picrorrhiza kurroa which represent a synergistic combination of Extracts. Preferred such combinations include Extracts formed by the combination of the following pairs of Extracts: Extracts 16 and 17; 17 and 33; 10 and 17; 30 and 33; 30 and 32; 32 and 33; 14 and 18; 17 and 18; and 17 and 54, and combinations of Extracts or compositions substantially similar to any of these combinations. Particularly preferred combinations are Extracts formed by the combination of the following pairs of Extracts: Extracts 30 and 33; 30 and 32; 32 and 33; and 14 and 18; and combinations of Extracts or compositions substantially similar to any of these combinations.

In a still further aspect the present invention provides the use of one or more of the compositions, mixtures or Extracts described herein in the manufacture of a medicament for the treatment of disease.

It will be appreciated that compositions which have the problem Component(s) (e.g. inflammatory components) and/or Komponent(s) removed and which include additional active Extracts are also within the scope of the invention.

Thus, according to the present invention in a further aspect there is provided a pharmaceutical composition or food supplement comprising an Extract of natural Ginkgo biloba and an Extract of natural Picrorrhiza kurroa each of which Extracts, gives a substantially positive result in a neutrophil oxidative burst assay (for example, a value more positive than +10%, in the test set out in section 3 below); and which has substantially no Components(s) which give a substantially negative result in a neutrophil oxidative burst assay; provided that the Extract of natural Picrorrhiza kurroa does not contain apocynin or apocynin derivative as the sole pharmacologically active species.

According to the present invention in a further aspect there is provided a pharmaceutical composition or food supplement comprising an Extract of natural Ginkgo biloba and an Extract of natural Picrorrhiza kurroa each of which Extracts, gives a substantially positive result in a neutrophil oxidative burst assay (for example, a value more positive than +10%, in the test set out in section 3 below); and which has substantially no Komponents(s); provided that the Extract of natural Picrorrhiza kurroa does not contain apocynin or apocynin derivative as the sole pharmacologically active species.

Preferably, the composition has been treated to remove (or lower the proportion of) one or more Component(s) or Komponent(s).

The compositions, preparations and methods according to the invention are useful as (or in the manufacture of) pharmaceutical preparations for the treatment of human patients, and/or as (or in the manufacture of) veterinary preparations for the treatment of non human animals and, and/or as food supplements because they have increased anti-inflammatory potency (and/or reduced side effects, e.g. pro-inflammatory potency) as discussed below and e.g. shown in the Assay in section 3 below.

The Assay indicates that the composition(s) and/or preparation(s) are suitable for use in the treatment or amelioration or relief of inflammatory disease. Preferably, they are used for the treatment or amelioration or relief of inflammatory respiratory disease. “Inflammatory respiratory disease” includes respiratory diseases such as asthma, allergic airways disease and emphysema (e.g. hereditary emphysema), symptoms of allergy manifested in the respiratory system, exercise induced asthma and chronic obstructive pulmonary disease (COPD) (bronchitis) and non allergenic respiratory diseases of bacterial, viral or fungal origins. The preparation may also be used to treat inflammatory diseases such as inflammatory joint disease, arthritis and rheumatoid osteoarthritis, (atopic) dermatitis, leishmaniasis and/or inflammatory diseases of the gastrointestinal tract, such as ulcers (including stomach ulcers), gastric ulcer syndrome, ulcerative collitis, coeliac disease, irritable bowel syndrome, irritable bowel disease and Crohn's disease. The preparations may also be used to prevent platelet induced blood clotting, and thus are suitable for use in the treatment, management and/or prevention of conditions caused by platelet induced blood clotting (for example coronary artery disease, arterial clotting, PAD (peripheral arterial disease and stroke) The preparations and compositions may also be used in the treatment, management and/or prevention of thrombosis and cardiac related problems (for example ischaemia and blood flow problems, arrhythmias induced by experimental myocardial ischaemia, prevention or reduction of arteriolar spasm and problems caused by thrombus formation). The compositions and preparations may be used to treat, manage, or prevent nasal and/or ocular hayfever (allergic rhinitis) and other general manifestations such as reduced respiratory tract mucous production.

The compositions may be used as (or in the manufacture of) veterinary preparations for the treatment of non human animals, for example dogs, pigs, equine species, poultry and reared game birds such as pheasants. They may be used to treat inflammatory diseases such as seasonal pruritic dermatitis, laminitis, ecezmatous dermatitis, COPD, lameness, azoturia, dermatitis, rain scald, osteoathritis, hip dysplasia, leishamaniasis, equine gastroulcer syndrome, or the sequellae of any thereof.

It will also be understood that, amongst other things, the preparations of the invention are suitable for treatment or diminuition of the symptoms of allergic attacks or allergic reactions such as insect bites or stings. The preparations of the invention are suitable for treatment, amelioration or prevention of inflammatory gastrointestinal tract disorders. The preparations of the invention are suitable for treatment or prevention of inflammatory disease, e.g. inflammatory respiratory disease in animals, for example dogs and horses.

The compositions may also be used as a food supplement for humans and animals, e.g. a nutritional supplement with therapeutic effects. It may be used as a preventative measure against disease, or for treatment or management of a condition.

Treatment using preparations of the invention may lead to considerable benefits in general health, such as up to a 95% reduction in symptoms of asthma and other more general manifestations, such as increased wellness and happiness, colour (indicative of improved oxygenation), and, in children, apparent increased growth and development. There may also be a significant reduction in the amount of crises and thus a decreased need for hospital or out-patient treatment.

“Treatment using preparations of the invention” should be taken to mean both administration of the preparation at a preventative or maintenance dose with the aim of prophylaxis (that is preventing or at least reducing the frequency of attacks and therefore maintaining a level of health, and/or preventing the development of a condition), and treatment at a (generally higher) “therapeutic level” to alleviate chronic attacks or crisis symptoms which if untreated may lead to hospitalisation. Thus the preparation may remove or reduce the need for treatments considered harmful such as corticosteroids, especially oral corticosteroid treatments. However, the preparations/compositions may be administered in combination with conventional asthma treatments (beta androgenic drugs such as, for example, salbutamol, oxygen therapy, corticosteroids, breathing techniques etc) without ill effects, and may enable the dose of conventional treatment to be reduced (dose sparing).

Preferred pharmacutical compositions and food supplements further comprise an antioxidant which inhibits the activation of the Nuclear Factor kappa B (NF-kB) cascade and/or interferes with the arachidionic acid cascade, for example apocynin or apocynin derivative.

Apocynin interferes with arachidonic acid cascade, increases glutathione synthesis, and is a neutrophil oxidative burst antagonist.

Pharmaceutical compositions or food supplements may also comprise other antioxidants which inhibit the activation of the Nuclear Factor kappa B (NF-kB) cascade and/or interfere with the arachidionic acid cascade, for example 3,5-Dimethoxy-4 hydroxyacetophenone (Acetosyringenin).

Preferably, the pharmaceutical composition (or preparation) or food supplement further comprises an agent which enhances lipid solubility of the preparation. This gives rise to better absorption and hence better bioavailability, especially by the oral route. Preferred agents which enhance lipid solubility are sources of pharmaceutically acceptable surfactants and/or fatty acids, for example phosphatidylcholine (lecithin).

The preparation (pharmaceutical composition or food supplement) may further comprise additional components such as pharmaceutically conventional carriers, diluents, flavourants, emulsifiers and stabilisers. Preferably the preparation further comprises one or more of the following:

i) an agent to enhance the immune system, for example lactoferrin which has anti-viral, antibacterial and/or anti-oxidant effects;

iii) a source, for example a natural source, of vitamins, minerals and/or amino acids, for example chlorella and/or bee pollen;

iv) a source of trace elements, for example fucus vesiculosus; and/or

v) taste masking agents, for example yoghurt, fruit juice, honey and syrup.

It is preferred that the preparation is administered orally, for example in pill or capsule form, although it is possible, to use other known conventional administration techniques.

The optimum proportions of the various active Extracts in the compositions may vary within wide limits and will of course vary with the patient and the condition to be treated. However we prefer a ratio (weight for weight) of active Extract of natural Ginkgo biloba: Extract of natural Picrorrhiza kurroa: antioxidant e.g. apocynin (if present) of between 75:100:1 to 0.75:1:100, preferably between 37.5:50:1 and 0.75:1:20; preferably 3:4:20.

A preferred daily dose of active Extract (according to the invention) for a human is from 10 μg/kg to 10 mg/kg. A preferred daily dose of antioxidant, e.g. apocynin, (in those compositions which include this) is between from 60 μg to 20 mg/kg. Preferably these are split twice daily, 2/5 dose in the morning, 3/5 in the afternoon/evening.

A preferred unit dose of Extract of natural Picrorrhiza kurroa (Picrorrhiza kurroa) is, for an adult human (70 kg), between 175 μg and 700 mg. A preferred unit dose of Extract of natural Picrorrhiza kurroa (Picrorrhiza kurroa in the mixture) is, for a child (20 kg), between 50 μg and 200 mg. The preferred unit doses of Extract of natural Ginkgo biloba (Ginkgo biloba in the mixture) are approximately 75% of these.

A preferred unit dose of antioxidant (e.g. apocynin) is, for an adult human (70 kg), between 1050 μg and 140 mg. A preferred unit dose of Extract of natural Picrorrhiza kurroa (Picrorrhiza kurroa) is, for a child (20 kg), between 300 kg and 400 mg.

The compositions (and preparations) of the invention may be used as a sole treatment. They may also be used alongside conventional medicines (e.g. anti-allergics such as steroids and antihistamines which have unwanted side effects); this may lead to a reduction in the dose of conventional medicine required and thus a reduction in likelihood/occurunce of the side effects. A reduction of side effects of a conventional or known therapeutic agent (for example the side effects of anti-allergic agents) or simply enabling a lower dose of therapeutic agent to be administered without loss of pharmacological effect, during treatment of human or animal patients being treated is known as “dose sparing”.

Thus, according to the invention in a still further aspect there is provided a method of dose sparing a conventional therapeutic agent comprising the step of administering to the patient pharmaceutical compositions as described herein.

The preparation may be administered at the same time as the conventional or known therapeutic agent (for example the anti-allergic agent) or at a different time, by the same administration route, or by a different administration route.

The description relates (in places) to asthma but it will be understood that the preparations of the invention are suitable for treatment or amelioration or prevention of other diseases, especially other inflammatory diseases, as discussed above.

### DETAILED DESCRIPTION OF THE INVENTION

The following demonstrates the separation and analysis of the Extracts of natural Ginkgo biloba and natural Picrorrhiza kurroa, that is separation of the Extracts described above. The first stage is production of a “Fraction Library” of extracts.

1. Picrorrhiza Kurroa

1.1 Methanol Extraction and Dowex 50 (H⁺) Column

Dried, ground, plant material natural Picrorrhiza kurroa of the above species (180 g) was extracted with methanol (600 cm³ Methanol, stirred and left to steep for 16 hours at Room temperature). The plant material was then separated from the extract by filtration. The plant material was dried, using a rotary evaporator, ready for further extraction work. Water (600 cm³) was then added to the methanol extract, which was then passed down a Dowex 50 (H⁺) column (250 cm³), collecting unbound material (BR000001) for further work. The column was subsequently washed with 50:50 Ethanol-Water (500 cm³). The column was then eluted with 2N NH₄OH (1000 cm³). The NH₄OH was removed from the eluant by rotary evaporation to yield (BR000002).

1.2 HP20 Scavenge and Flash Chromatography

The unbound Dowex 50 (H⁺) fraction (BR000001) was passed through a HP20 cartridge (Biotage 75S) collecting unbound material. The cartridge was then washed with 25:75 Methanol-Water (500 cm³). The unbound and wash were combined (BR000003). The cartridge was then eluted with 10:90 Methanol-Acetone (1000 cm³) (BR000004) (Yield: 14 g).

BR000004 (4 g) was dried onto silica (12 g) (that is loaded onto silica and the solvent removed) and packed into a SIM (Sample Injection Module), connected to a Biotage 40M Silica Cartridge, eluting with the following solvent systems (in the following sections the solvent percentages are by volume):

| | | Ethyl Acetate | |
|---|---|---|---|
| Fraction | Hexane (%) | (%) | Methanol (%) |
| BR000008 | 75 | 25 | |
| BR000009 | 25 | 75 | |
| BR000010 | | 100 | |
| BR000011 | | 90 | 10 |
| BR000012 | | 50 | 50 |
| BR000013 | | | 100 |

1.3 Dichloromethane Extraction and Flash Chromatography

The dried plant material, remaining after step 1.1 (140 g), was extracted with dichloromethane (DCM) (4500 cm³) using Soxhlet apparatus to yield sample BR000005. The extract was bound onto HP20 resin, packed into a SIM (Sample Injection Module) and connected to a Biotage 75S HP20 Cartridge, which was eluted with 10:90 Methanol-Acetone (4000 cm³) (BR000006) (Yield: 3 g), followed by Acetone (1000 cm³) (BR000007).

BR000006 (3 g) was dried onto silica (12 g) and packed into a SIM (Sample Injection Module), connected to a Biotage 40M Silica Cartridge, eluting with the following solvent systems:

| | Hexane | Ethyl Acetate | Methanol |
|---|---|---|---|
| Fraction | (%) | (%) | (%) |
| BR000014 | 100 | | |
| BR000015 | 75 | 25 | |
| BR000016 | 50 | 50 | |
| BR000017 | 25 | 75 | |
| BR000018 | | 100 | |
| BR000019 | | 90 | 10 |

1.4 Methanol Extraction and Semi Prep HPLC

Picrorrhiza kurroa (2.5 g dried plant material) was extracted with methanol three times (each time 20 cm³ Methanol added with shaking, the mixture allowed to settle for 15 minutes and filtered); the filtrates were combined and dried down (Yield 650 mg). 100 mg of the dried methanol extract was dissolved in methanol (1 cm³) and applied to a semi-prep HPLC column (25×100 mm), eluting with the following solvent systems (using a linear gradient):

| Time | | | | |
|---|---|---|---|---|
| Min | % A | % B | % C | % D |
| 0 | 80 | 10 | 10 | 0 |
| 2 | 80 | 10 | 10 | 0 |
| 14 | 10 | 80 | 10 | 0 |
| 18 | 10 | 80 | 10 | 0 |
| 20 | 0 | 0 | 0 | 100 |
| 22 | 10 | 80 | 10 | 0 |
| 24 | 80 | 10 | 10 | 0 |
| 28 | 80 | 10 | 10 | 0 |

A=Water; B=Acetonitrile; C=Acetonitrile+0.001% Trifluoroacetic acid; and D=Acetone. Flow Rate: 12 cm³min⁻¹. Fraction collector: 4-minute delay after injection followed by 18×30 second fractions (BR000020-BR000037)

2 Ginkgo Biloba

2.1 Methanol Extraction and Dowex 50 (H⁺) Column

Ginko extract (commercially available dried powder natural Ginko biloba) (50 g) was dissolved in methanol (500 cm³), water (500 cm³) was then added to the methanol extract which was then passed down a Dowex 50 (H⁺) column (250 cm³), collecting unbound material (BR000038) for further work. The column was subsequently washed with 50:50 Ethanol-Water (500 cm³). The column was then eluted with 2N NH₄OH (1000 cm³). The NH₄OH was removed by rotary evaporation to yield (BR000039).

2.2 HP20 Scavenge and Flash Chromatography

The unbound Dowex 50 (H⁺) fraction (BR000038) was passed through a Biotage 75S HP20 cartridge collecting unbound material. The cartridge was then washed with 25:75 Methanol-Water (500 cm³). The unbound and wash were combined (BR000040). The cartridge was then eluted with 10:90 Methanol-Acetone (1000 cm³) (BR0000641) (Yield: 16 g).

BR000041 (4 g) was dried onto silica (12 g) and packed into a SIM, connected to a Biotage 40M Silica Cartridge, eluting with the following solvent systems:

| | Hexane | Ethyl | Methanol |
|---|---|---|---|
| Fraction | (%) | Acetate (%) | (%) |
| BR000042 | 75 | 25 | |
| BR000043 | 25 | 75 | |
| BR000044 | | 100 | |
| BR000045 | | 90 | 10 |
| BR000046 | | 50 | 50 |
| BR000047 | | | 100 |

2.3 Methanol Extraction and Semi Prep HPLC

Ginko biloba extract (100 mg) was dissolved in methanol (1 cm³) and applied to a semi-prep HPLC column (25×100 mm), eluting with the following solvent system:

| Time | % A | % B | % C | % D |
|---|---|---|---|---|
| 0 | 80 | 10 | 10 | 0 |
| 2 | 80 | 10 | 10 | 0 |
| 14 | 10 | 80 | 10 | 0 |
| 18 | 10 | 80 | 10 | 0 |
| 20 | 0 | 0 | 0 | 100 |
| 22 | 10 | 80 | 10 | 0 |
| 24 | 80 | 10 | 10 | 0 |
| 28 | 80 | 10 | 10 | 0 |

A=Water; B=Acetonitrile; C=Acetonitrile+0.001% Trifluoroacetic acid; and D=Acetone. Flow Rate: 12 cm³min⁻¹. Fraction collector: 4-minute delay after injection followed by 19×30 second fractions (BR000048-BR000066).

3 Dihydrorhodamine Flow Cytometric Assay for the Effect of Plant Extracts on Neutrophil Respiratory Burst on Peripheral Blood

An aliquot (200 μg) of every fraction (obtained by the methods described above) was prepared for assay. Tables 1 and 2 summarise the fractions supplied. The left hand column (Extract number) will be the number used to denote that fraction in subsequent studies and corresponds to the Fraction numbers (“BR0000n”) used above reduced to 1,2, etc., by removal of the “BR00000”.

3.1 Background of Assay

Neutrophils kill organisms they have ingested by exposing them to chemicals known as reactive oxygen species (eg. hydrogen peroxide and hydroxyl ions). These are generated by a process known as the neutrophil respiratory burst. Respiratory burst and the consequent superoxide anion production can be artificially induced when neutrophils of fresh peripheral blood are stimulated with PMN (Phorbol Myristate Acetate). Respiratory burst occurs when stimulated neutrophils convert molecular oxygen to oxygen radicals through the activation of NADPH-oxidase. In the dihydrorhodamine (DHR) flow cytometric assay, the respiratory burst is used to produce fluorescent molecules that can be detected by flow cytometry. Specifically, the non-fluorescent dye dihydrorhodamine 123 is oxidised by the reactive oxygen radicals to convert into rhodamine, which fluoresces green when exposed to the argon laser with a wavelength of 488 nm. The extent of respiratory burst product can be further quantified using the mean fluorescence intensity of the FL-1 channel (MFI).

The main purpose of the assay (which is well known in the art) is to detect patients who have abnormal neutrophil function. One such condition is Chronic Granulomatous Disease (CGD) in which patients' neutrophils are incapable of producing respiratory burst. Normal individuals will produce rhodamine from DHR in all stimulated neutrophils. In patients with homozygous CGD the neutrophils cannot produce rhodamine, thus none of their neutrophils will produce fluorescence. Carriers of CGD (heterozygous) can also be detected by this assay, where half of their neutrophils are normal and half have no respiratory burst. Thus two populations of cells can be detected by flow cytometry—one population which fluorescence and one that does not.

Thus, the test is a measure of the neutrophil oxidative burst reaction and is used in a clinical setting to diagnose chronic granulomatous disease. Patients with this disease have defective neutrophils and a different neutrophil oxidative burst reaction to normal patients. The test was modified (for the present purpose) by the addition of the Extract under investigation to the blood sample. In the following, a positive assay result (that is, a positive number in the Assay Result column of Tables 1 and 2) indicates anti-inflammatory activity, and a negative result indicates pro-inflammatory activity. A result of anti-inflammatory activity indicates possible utility in treatment of inflammatory disease, for example inflammatory respiratory disease such as asthma.

3.2 Method

Dihydrorhodamine (DHR) 123 (Sigma order no. D 1054) was prepared as a stock solution by dissolving 1 mg DHR in 1 ml dimethyl sulphoxide (DMSO) and stored as 50 μl aliquots at −70° C. A working solution of DHR was prepared by adding 20 μl stock DHR solution to 650 μl PBS. The solution was kept in the dark.

Phorbol myrisate acetate (PMA, Sigma order no. P 8139). was prepared as a stock solution by dissolving 1 mg PMA in 1 ml dimethyl sulphoxide (DMSO) and stored as 50 μl aliquots at −20° C. Intermediate strength PMA solution (50 μM) was prepared by adding 30 μl stock PMA solution to 970 μl Phosphate buffered saline (PBS). Diluting 100 μl of this solution 1:10 with PBS provides working PMA solution (5 μM) {100 μl+900 μl PBS).

FACS lysing solution was diluted 1:10 using deionised water.

1% Paraformaldehyde (PFA) was added to Phosphate Buffered Saline and kept at 52° C. for three hours or until completely dissolved.

100 μl blood was-added to a tube, to which was added 25 μl of plant extract (stock of 400 μg/ml) and 25 μl of PMA. A tube was prepared for each extract. The control tube contains only blood (heparinized blood from a normal subject) and 25 μl PMA. Any background fluorescence (as a further control) is estimated using a tube containing only blood and DHR.

All tubes were incubated in a 37° C. incubator for 15 minutes. 25 μl DHR solution was added to each tube, and these were incubated at 37° C. for exactly 7 minutes. Removal of all red cells was effected using FACS lysing solution, with subsequent centrifugation and washing in PBS (twice) to remove lysed red cells. Leucocytes were vortexed and fixed in 1% paraformaldehyde in PBS.

FACSCalibur flow cytometer was standardised (using Calibrite fluorescent beads and running the FACSComp program) and acquisition and analysis performed (by opening the DHR assay icon and the DHR assay setting; identifying neutrophils on dot plot of forward scatter (FSC) and side scatter (SSC); adjusting FSC and SSC so that neutrophil “cloud” is in centre of screen; detecting Rhodamine fluorescence on FL1 at 530 nm (a histogram of FL1 and adjusted photomultiplier gains was displayed so that unstimulated cells were collected in the first decade and stimulated cells collected the in second or third decade); and acquiring events/counts from the stimulated tube, after adjusting the data collection parameters to count 5500 gated events.

The results are presented in the attached Tables 1 and 2.

3.3 Results of Natural Picrorrhiza Kurroa Extracts

TABLE 1

| Extract | | Assay |
|---|---|---|
| (Number) | Extraction procedure | Result (%) |
| 1 | Polar unbound | −31 |
| 2 | Polar bound | −15 |
| 3 | HP20 Scavenge unbound | −13 |
| 4 | HP20 Scavenge bound | −7 |
| 5 | DCM Extract | 4 |
| 6 | “Cleaned up” DCM Extract | 10 |
| 7 | Acetone wash | −8 |
| 8 | HP20 Scavenge Flash Fraction | −3 |
| 9 | HP20 Scavenge Flash Fraction | 3 |
| 10 | HP20 Scavenge Flash Fraction | 21 |
| 11 | HP20 Scavenge Flash Fraction | 9 |
| 12 | HP20 Scavenge Flash Fraction | 5 |
| 13 | HP20 Scavenge Flash Fraction | 3 |
| 14 | DCM Flash Fraction | 21 |
| 15 | DCM Flash Fraction | 12 |
| 16 | DCM Flash Fraction | 27 |
| 17 | DCM Flash Fraction | 48 |
| 18 | DCM Flash Fraction | 35 |
| 19 | DCM Flash Fraction | −24 |
| 20 | Semi prep HPLC Fraction | −11 |
| 21 | Semi prep HPLC Fraction | −3 |
| 22 | Semi prep HPLC Fraction | −2 |
| 23 | Semi prep HPLC Fraction | 10 |
| 24 | Semi prep HPLC Fraction | 5 |
| 25 | Semi prep HPLC Fraction | 8 |
| 26 | Semi prep HPLC Fraction | −11 |
| 27 | Semi prep HPLC Fraction | −18 |
| 28 | Semi prep HPLC Fraction | −5 |
| 29 | Semi prep HPLC Fraction | 15 |
| 30 | Semi prep HPLC Fraction | 21 |
| 31 | Semi prep HPLC Fraction | 18 |
| 32 | Semi prep HPLC Fraction | 21 |
| 33 | Semi prep HPLC Fraction | 24 |
| 34 | Semi prep HPLC Fraction | 8 |
| 35 | Semi prep HPLC Fraction | 7 |
| 36 | Semi prep HPLC Fraction | 1 |
| 37 | Semi prep HPLC Fraction | 1 |

Extract 6, the “cleaned up” DCM (dichloromethane) extract, was cleaned by passing (filtering) through a HP20 column to remove chlorophyll degradation products and other material which would bind irreversibly to the HPLC column.

3.4 Results of Natural Ginkgo Biloba Extracts

TABLE 2

| Extract | | Assay |
|---|---|---|
| (Number) | Extraction procedure | Result (%) |
| 38 | Polar unbound | 32 |
| 39 | Polar bound | 10 |
| 40 | HP20 Scavenge unbound | 30 |
| 41 | HP20 Scavenge bound | 49 |
| 42 | DCM Extract | 40 |
| 43 | Cleaned up DCM Extract (see | 46 |
| | Extract 6 for cleaning | |
| | process) | |
| 44 | Acetone wash | 59 |
| 45 | HP20 Scavenge Flash Fraction | 49 |
| 46 | HP20 Scavenge Flash Fraction | 14 |
| 47 | HP20 Scavenge Flash Fraction | −6 |
| 48 | Semi prep HPLC Fraction | −4 |
| 49 | Semi prep HPLC Fraction | 14 |
| 50 | Semi prep HPLC Fraction | 36 |
| 51 | Semi prep HPLC Fraction | 21 |
| 52 | Semi prep HPLC Fraction | 21 |
| 53 | Semi prep HPLC Fraction | 44 |
| 54 | Semi prep HPLC Fraction | 72 |
| 55 | Semi prep HPLC Fraction | 55 |
| 56 | Semi prep HPLC Fraction | 32 |
| 57 | Semi prep HPLC Fraction | 22 |
| 58 | Semi prep HPLC Fraction | 52 |
| 59 | Semi prep HPLC Fraction | 65 |
| 60 | Semi prep HPLC Fraction | 51 |
| 61 | Semi prep HPLC Fraction | 49 |
| 62 | Semi prep HPLC Fraction | 36 |
| 63 | Semi prep HPLC Fraction | 32 |
| 64 | Semi prep HPLC Fraction | 48 |
| 65 | Semi prep HPLC Fraction | 15 |
| 66 | Semi prep HPLC Fraction | 9 |

The % error in the above is of the order of 10%.

Surprisingly, there are some negative results. All negative results in Table 1 and 2 indicate extracts that have pro-inflammatory activities or potencies; extracts which are therefore acting contra to the desired beneficial effects. The removal of one (or all) of these (called “Components” herein), from the ‘final product’ may have a positive therapeutic effect.

From these data a number of extracts (for example, Extracts 17, 18), which display promising activities can be selected. These are referred to as “Extracts” herein. These, and combinations of these, were selected from natural Picrorrhiza kurroa and natural Ginkgo biloba. Experiments were conducted with combinations of some above Extracts from natural Ginkgo biloba and natural Picrorrhiza kurroa to evaluate potential synergies and/or additive effects.

Experiments were conducted with combinations of Extracts (Table 3) and combinations of Extracts plus apocynin (Table 4) at 300 μg/ml final assay concentration. Sample numbers are arbitrary but Extract numbers are consistent with those in Tables 1 and 2 above.

TABLE 3
Combination Results without Apocynin

| | Extract | Extract | Extract | |
|---|---|---|---|---|
| Sample | 1 | 2 | 3 | Assay Result |
| 1 | | 10 | 14 | 15 |
| 2 | | 10 | 16 | 37 |
| 3 | | 10 | 17 | 52 |
| 4 | | 10 | 18 | 46 |
| 5 | | 10 | 41 | 32 |
| 6 | | 14 | 16 | 27 |
| 7 | | 14 | 17 | 48 |
| 8 | | 14 | 18 | 55 |
| 9 | | 16 | 17 | 46 |
| 10 | | 16 | 18 | 34 |
| 11 | | 17 | 18 | 66 |
| 12 | | 30 | 32 | 53 |
| 13 | | 30 | 33 | 59 |
| 14 | | 32 | 33 | 52 |
| 15 | | 17 | 33 | 68 |
| 16 | | 17 | 41 | 71 |
| 17 | | 17 | 50 | 71 |
| 18 | | 17 | 54 | 82 |
| 19 | | 17 | 59 | 78 |
| 20 | | 17 | 64 | 54 |
| 21 | | 42 | 43 | 46 |
| 22 | | 42 | 44 | 51 |
| 23 | | 43 | 44 | 62 |
| 24 | 42 | 43 | 44 | 52 |
| 25 | | 50 | 54 | 78 |
| 26 | | 50 | 59 | 70 |
| 27 | | 50 | 64 | 65 |
| 28 | 50 | 54 | 59 | 79 |
| 29 | 50 | 54 | 64 | 72 |
| 30 | 50 | 59 | 64 | 63 |
| 31 | | 54 | 59 | 80 |
| 32 | | 54 | 64 | 71 |
| 33 | 54 | 59 | 64 | 74 |
| 34 | | 59 | 64 | 63 |

As can be seen from the Table, the most active (highest score) was a combination of Extract 17 and Extract 54 (“sample 18”). The top 20 performing Extract combinations were ranked as shown in Table 3a below:

TABLE 3a

| | | | | Extract | Assay |
|---|---|---|---|---|---|
| Rank | Sample | Extract 1 | Extract 2 | 3 | Result |
| 1 | 18 | 17 | 54 | | 82 |
| 2 | 31 | 54 | 59 | | 80 |
| 3 | 28 | 50 | 54 | 59 | 79 |
| 4 | 26 | 50 | 54 | | 78 |
| 5 | 19 | 17 | 59 | | 78 |
| 6 | 33 | 54 | 59 | 64 | 74 |
| 7 | 29 | 50 | 54 | 64 | 72 |
| 8 | 32 | 54 | 64 | | 71 |
| 9 | 16 | 17 | 41 | | 71 |
| 10 | 17 | 17 | 50 | | 71 |
| 11 | 26 | 50 | 59 | | 70 |
| 12 | 15 | 17 | 33 | | 68 |
| 13 | 11 | 17 | 18 | | 66 |
| 14 | 27 | 50 | 64 | | 65 |
| 15 | 30 | 50 | 59 | 64 | 63 |
| 16 | 34 | 59 | 64 | | 63 |
| 17 | 23 | 43 | 44 | | 62 |
| 18 | 13 | 30 | 33 | | 59 |
| 19 | 8 | 14 | 18 | | 55 |
| 20 | 20 | 17 | 64 | | 54 |

TABLE 4
Combination Results with Apocynin
In these assays a calibration curve of the effect of the
concentration of apocynin in this assay was created.
Based upon this curve, a concentration of apocynin of
300 ug/ml was chosen, as the concentration resulting
in 45% inhibition, for addition to each of the
combinations described in Table 3. The assay results
in the presence of this added concentration of apocynin
are presented in the Table below.

| Sample | Extract 1 | Extract 2 | Extract 3 | Assay Result |
|---|---|---|---|---|
| 1 | | 10 | 14 | 64 |
| 2 | | 10 | 16 | 70 |
| 3 | | 10 | 17 | 72 |
| 4 | | 10 | 18 | 69 |
| 5 | | 10 | 41 | 75 |
| 6 | | 14 | 16 | 66 |
| 7 | | 14 | 17 | 69 |
| 8 | | 14 | 18 | 64 |
| 9 | | 16 | 17 | 76 |
| 10 | | 16 | 18 | 65 |
| 11 | | 17 | 18 | 71 |
| 12 | | 30 | 32 | 67 |
| 13 | | 30 | 33 | 70 |
| 14 | | 32 | 33 | 71 |
| 15 | | 17 | 33 | 73 |
| 16 | | 17 | 41 | 74 |
| 17 | | 17 | 50 | 75 |
| 18 | | 17 | 54 | 71 |
| 19 | | 17 | 59 | 77 |
| 20 | | 17 | 64 | 67 |
| 21 | | 42 | 43 | 70 |
| 22 | | 42 | 44 | 81 |
| 23 | | 43 | 44 | 72 |
| 24 | 42 | 43 | 44 | 77 |
| 25 | | 50 | 54 | 74 |
| 26 | | 50 | 59 | 77 |
| 27 | | 50 | 64 | 74 |
| 28 | 50 | 54 | 59 | 85 |
| 29 | 50 | 54 | 64 | 72 |
| 30 | 50 | 59 | 64 | 79 |
| 31 | | 54 | 59 | 82 |
| 32 | | 54 | 64 | 82 |
| 33 | 54 | 59 | 64 | 76 |
| 34 | | 59 | 64 | 74 |
| 35 | Apocynin | | | 48 |

The assays conducted in all the ‘combination experiments’ (Tables 3, 4) were conducted with the same total concentration of material, but the concentration of a specific extract would be ½ or ⅓ of that in the ‘stand alone’ assays (Tables 1, 2).

Using this information a calculated ‘Predicted Combination Result’ could be made calculated based on adding the contribution of the stand alone value for each of the components of the combination mixture (adjusted to account for the difference in concentration used in the stand alone assays). This is based on several assumptions, including assay linearity and assay response linearity from the tested material. This could then be compared with the “Actual Combination Assay result”—which is the result obtained by performing the Assay on a real sample which combines the components.

Table 5 summarises the data in a single table including; raw data from the combinations (Table 3, 4), calculated ‘Predicted Combination Result’, actual “Actual Combination Assay results”, synergistic effects and the apparent effect of apocynin.

| | | | | | | | Predicted | Actual | | Predicted | |
|---|---|---|---|---|---|---|---|---|---|---|---|
| | | | | Extract 1 | Extract 2 | Extract 3 | Combination | Combination | Potential | Combination | |
| | Extract | Extract | Extract | Assay | Assay | Assay | Result (without | Assay result (no | ‘synergistic | result (plus | Effect of |
| Sample | 1 | 2 | 3 | Result | Result | Result | apocynin) | apocynin) | effect’ | apocynin) | apocynin |
| 1 | | 10 | 14 | | 21 | 21 | 21.00 | 15 | -6.00 | 64 | 49 |
| 2 | | 10 | 16 | | 21 | 27 | 24.00 | 37 | 13.00 | 70 | 33 |
| 3 | | 10 | 17 | | 21 | 48 | 34.50 | 52 | 17.50 | 72 | 20 |
| 4 | | 10 | 18 | | 21 | 35 | 28.00 | 46 | 18.00 | 69 | 23 |
| 5 | | 10 | 41 | | 21 | 49 | 35.00 | 32 | -3.00 | 75 | 43 |
| 6 | | 14 | 16 | | 21 | 27 | 24.00 | 27 | 3.00 | 66 | 39 |
| 7 | | 14 | 17 | | 21 | 48 | 34.50 | 48 | 13.50 | 69 | 21 |
| 8 | | 14 | 18 | | 21 | 35 | 28.00 | 55 | 27.00 | 64 | 9 |
| 9 | | 16 | 17 | | 27 | 48 | 37.50 | 46 | 8.50 | 76 | 30 |
| 10 | | 16 | 18 | | 27 | 35 | 31.00 | 34 | 3.00 | 65 | 31 |
| 11 | | 17 | 18 | | 48 | 35 | 41.50 | 66 | 24.50 | 71 | 5 |
| 12 | | 30 | 32 | | 21 | 21 | 21.00 | 53 | 32.00 | 67 | 14 |
| 13 | | 30 | 33 | | 21 | 24 | 22.50 | 59 | 36.50 | 70 | 11 |
| 14 | | 32 | 33 | | 21 | 24 | 22.50 | 52 | 29.50 | 71 | 19 |
| 15 | | 17 | 33 | | 48 | 24 | 36.00 | 68 | 32.00 | 73 | 5 |
| 16 | | 17 | 41 | | 48 | 49 | 48.50 | 71 | 22.50 | 74 | 3 |
| 17 | | 17 | 50 | | 48 | 36 | 42.00 | 71 | 29.00 | 75 | 4 |
| 18 | | 17 | 54 | | 48 | 72 | 60.00 | 82 | 22.00 | 71 | -11 |
| 19 | | 17 | 59 | | 48 | 65 | 56.50 | 78 | 21.50 | 77 | -1 |
| 20 | | 17 | 64 | | 48 | 48 | 48.00 | 54 | 6.00 | 67 | 13 |
| 21 | | 42 | 43 | | 40 | 46 | 43.00 | 46 | 3.00 | 70 | 24 |
| 22 | | 42 | 44 | | 40 | 59 | 49.50 | 51 | 1.50 | 81 | 30 |
| 23 | | 43 | 44 | | 46 | 59 | 52.50 | 62 | 9.50 | 72 | 10 |
| 24 | 42 | 43 | 44 | 40 | 46 | 59 | 48.33 | 52 | 3.67 | 77 | 25 |
| 25 | | 50 | 54 | | 36 | 72 | 54.00 | 78 | 24.00 | 74 | -4 |
| 26 | | 50 | 59 | | 36 | 65 | 50.50 | 70 | 19.50 | 77 | 7 |
| 27 | | 50 | 64 | | 36 | 48 | 42.00 | 65 | 23.00 | 74 | 9 |
| 28 | 50 | 54 | 59 | 36 | 72 | 65 | 57.67 | 79 | 21.33 | 85 | 6 |
| 29 | 50 | 54 | 64 | 36 | 72 | 48 | 52.00 | 72 | 20.00 | 72 | 0 |
| 30 | 50 | 59 | 64 | 36 | 65 | 48 | 49.67 | 63 | 13.33 | 79 | 16 |
| 31 | | 54 | 59 | | 72 | 65 | 68.50 | 80 | 11.50 | 82 | 2 |
| 32 | | 54 | 64 | | 72 | 48 | 60.00 | 71 | 11.00 | 82 | 11 |
| 33 | 54 | 59 | 64 | 72 | 65 | 48 | 61.67 | 74 | 12.33 | 76 | 2 |
| 34 | | 59 | 64 | | 65 | 48 | 56.50 | 63 | 6.50 | 74 | 11 |

The combinations showing the greatest synergistic effects (i.e. where the Actual Combination Assay Result is rather greater than the Predicted Combination Assay Result) are:

| | | | | | Synergistic |
|---|---|---|---|---|---|
| | | | | | Effect on |
| | | | | | Actual |
| | | | | | Assay |
| | | | | | Result over |
| Rank | Sample | Extract 1 | Extract 2 | Extract 3 | Anticipated |
| 1 | 13 | 30 | 33 | | + 36.5 |
| 2 | 12 | 30 | 32 | | +32 |
| 3 | 14 | 32 | 33 | | +29.5 |
| 4 | 16 | 17 | 41 | | +29 |
| 5 | 8 | 14 | 18 | | +27 |
| 6 | 11 | 17 | 18 | | +24.5 |
| 7 | 24 | 42 | 43 | 44 | +24 |
| 8 | 27 | 50 | 64 | | +23 |
| 9 | 16 | 17 | 41 | | +22.5 |
| 10 | 18 | 17 | 54 | | +22 |

From the data presented here it has been possible to focus on the Extracts of natural Ginkgo biloba and Extracts of natural Picrorrhiza kurroa which demonstrate activity either alone or in combination with one another with and/or without the addition of apocynin. In addition those Extracts demonstrating most ‘synergistic’ potential have also been selected. The study has allowed selection of 15 extracts (“Components”) which possess activities detrimental to the desired therapeutic activity, such activities are observed in (from Picrorrhiza kurroa) extracts 1, 2, 3, 4, 7, 8, 19, 20, 21, 22, 26, 27 and 28 and (from Ginkgo biloba) extracts 47 and 48. The study has also allowed selection of at least 12 extracts which possess desirable therapeutic activities, such activities are observed in natural Picrorrhiza kurroa extracts 10, 14, 17, 18, 30, 32 and 33 and natural Ginkgo biloba extracts 41, 50, 54, 59 and 64.

5 HPLC Analysis—Characterisation of Fractions

The above described work has resulted in chemical profiling of methanol extracts of the plant Picrorrhiza kurroa and of Ginkgo biloba extract, fractionation of extracts of these plants by various chromatographic methods, and assay of the fractions (and of selected combinations of fractions) for effect on neutrophil oxidative burst. Based on the results, twelve Extracts having desirable therapeutic activities and fifteen Components with detrimental activities have been shown in Tables 1 and 2. Characteristics of these highlighted fractions (the fraction numbers are those used in the Tables) are summarised below. The extracts of interest were analysed by HPLC with photodiode array (PDA) and evaporative light scattering (ELS) detectors.

Extracts with Desirable (Anti-Inflammatory) Activity

We believe that the following give rise to desirable activity:
Fatty acids—Extract 14;
Iridoid glycosides—Extract 30;
Flavonoids or flavonoid glycosides; Ginkgolides—Extract 41;
Quercitrin (the rhamnoside of the flavonoid quercetin—Extract 54; and
The flavonoid campherol—Extract 64.

Extracts of natural Picrorrhiza kurroa which have log P of between −1.9 and 4.9, especially between 1 and 2.5. These include apocynin, the picrosides, for example one or more of picroside I, III and IV, arvenin-III Root, picrorhizin, veronicoside, picroside II, kutkoside, pikuroside, scroside A scroside B, scroside C, plantamajoside, picein and androsin.

Extracts of natural Ginkgo biloba which have log P of between −1.9 and 4.9, especially between 1 and 2.5. These include quercetin, quercetin-3-0-rutinoside and/or quercetin-3-rhamnoglucoside, 6-hydroxykynurenic-acid, acacetin, apigenin, bilobalide, d-catechin, d-gallocatechin, isorhamnetin, kaempferol, 1-epicatechin, 1-epigallocatechin, luteolin, quercetin, quercetin-3-0-rutinoside, quercetin-3-rhamnoglucoside, sequoyitol, shikimic-acid, and tricetin leaf.

Fractions (“Extracts”) With Inflammatory Activity

We believe that the following give rise to Inflammatory Activity activity:
Extract 2—sugars;
Extract 8—fatty acids; and
Extract 19—polyunsaturated fatty acids.
Components (extracts) of natural Picrorrhiza kurroa having log P of −2 or more negative, where P is the Partition Ratio between Octanol/Water—Examples of such Components are catalpol, verminoside and aucubin.
Components (extracts) of natural Picrorrhiza kurroa log P of 5 or greater—Examples of such Components include tripalmitin.

Components (extracts) of natural Ginkgo biloba having log P of 5 or greater. Examples of such Components include 10-heptacosanol, 10-nonacosanol, 13-nonacosanol, amentoflavone, bilobetin, ginkgetin, Ginkgolic-acid, isoginkgetin, nonacosane, octacosanol-1, sciadopitysin, spinasterol and stigmasterol, and/or residues of any of these.

General

The data demonstrate that for both natural P. kurooa and natural Ginkgo biloba, methanol extracts can be fractionated to concentrate the active material by removing inactive or stimulatory (that is, inflammatory) components. The undesirable (inactive and/or inflammatory) material tends to be either the most polar or least polar material of natural Picrorrhiza kurroa, and the most polar material of Ginkgo biloba, which may include sugars and fats.

The assay results demonstrate that a larger number of the fractions possess inhibitory activity, but that for both Ginkgo boloba and Picrorrhiza kurroa activity can be localised by chromatographic fractions. The HPLC data indicate that several species of both natural plant extracts are active.

Activity in the natural Ginkgo biloba is concentrated by binding to and subsequently eluting from HP20 (BR000041) (Extract 41), i.e. separating from material that does not bind to HP20. The most active fraction from subsequent flash chromatography (BR000044) (Extract 44) is enriched in the peak with t_{R} 5.85 minutes compared with Extract 41; this peak is also predominant in BR000045 (Extract 45), the second most active flash fraction.

The results prove that there are multiple components contributing to inhibition of neutrophil oxidative burst in Extracts of natural P. kurroa and natural Ginkgo biloba, and demonstrate a degree of synergism when combining extracts.

The above demonstrate the basis for production of a botanical product, enriched in the active components inhibiting neutrophil oxidative burst, and to use HPLC analysis as a quality control check on batch-to-batch reproducibility. The removal of material not contributing to inhibition of neutrophil oxidative burst, or even stimulating it (witness negative assay results for some fractions), may reduce the risk of potential adverse side effects.

Example Preparations

1. Oral Formulation

The following reagents were mixed:

| | |
|---|---|
| apocynin | 100 mg |
| Extract 17 | 20 mg |
| Extract 54 | 15 mg. |

The mixture was mixed with vehicles, excipients, such as are known in the art and, using techniques which are well known in the art, prepared in form suitable for dosing, for example in capsule form for oral dose. The mixure may be used for treatment of asthma.

1. A pharmaceutical or food supplement comprising:
a mixture of Picrorrhiza kurroa and Ginkgo biloba which includes less of a component of natural Picrorrhiza kurroa than the amount of that component present in natural Picrorrhiza kurroa or less of a component of natural Ginkgo biloba than the amount of that component present in natural Ginkgo biloba;
which component, if isolated, gives a substantially negative result in a neutrophil oxidative burst assay (and is defined as “Component” in these claims);
provided that the Picrorrhiza kurroa does not contain apocynin or apocynin derivative as sole pharmacologically active species.

2. A pharmaceutical composition or food supplement according to claim 1 which includes less of a Component of natural Picrorrhiza kurroa than the amount of that Component present in natural Picrorrhiza kurroa.

3. A pharmaceutical composition or food supplement according to claim 1 which includes less of a Component of natural Ginkgo biloba than the amount of that Component present in natural Ginkgo biloba.

4. A pharmaceutical composition or food supplement according to claim 1 which includes the Component in an amount which is less than 70% w/w of the amount present in natural Picrorrhiza kurroa or natural Ginkgo biloba.

5. A pharmaceutical composition or food supplement according to claim 1 which includes the Component in an amount which is less than 10% w/w of the amount present in natural Picrorrhiza kurroa or natural Ginkgo biloba.

6. A pharmaceutical composition or food supplement according to claim 1 wherein the Component has log P of less than about −2.

7. A pharmaceutical composition or food supplement according to claim 1 wherein the Component has log P of greater than about +5.

8. A pharmaceutical composition or food supplement comprising:
a mixture of Picrorrhiza kurroa and Ginkgo biloba which includes less proportion of a component of natural Picrorrhiza kurroa than the amount of that component present in natural Picrorrhiza kurroa;
which component has log P of less than about −2 or of greater than about +5; provided that the Picrorrhiza kurroa does not contain apocynin or apocynin derivative as sole pharmacologically active species.

9. A pharmaceutical composition or food supplement according to claim 8 wherein the Component gives a substantially negative result in a neutrophil oxidative burst assay.

10. A pharmaceutical composition or food supplement according to claim 1 in which the physiologically active proportion of cucurbitacins is substantially zero.

11. A pharmaceutical composition or food supplement comprising:
a mixture of Picrorrhiza kurroa and Ginkgo biloba which includes less of a component of natural Picrorrhiza kurroa than the amount of that component present in natural Picrorrhiza kurroa;
which component is a cucurbitacin (and is defined as Komponent in these claims); provided that the Picrorrhiza kurroa does not contain apocynin or apocynin derivative as sole pharmacologically active species.

12. A pharmaceutical composition or food supplement according to claim 11 which includes the Komponent in an amount which is less than 10% w/w of the amount present in natural Picrorrhiza kurroa.

13. A pharmaceutical composition or food supplement according to claim 11 in which the physiologically active proportion of cucurbitacins is substantially zero.

14. A pharmaceutical composition or food supplement comprising a mixture of an Extract of natural Ginkgo biloga and an Extract of natural Picrorrhiza kurroa, each of which Extracts if isolated gives a substantially positive result in a neutrophil oxidative burst assay; provided that the Extract of natural picrorrhiza kurroa does not contain apocynin or apocynin derivative as the sole pharmacologically active species.

15. A pharmaceutical composition or food supplement according to claim 14 wherein the Extract of natural Ginkgo biloba has log P of between −1.9 and 4.9.

16. A pharmaceutical composition or food supplement according to claim 14 wherein the Extract of natural Picrorrhiza kurroa has log P of between −1.9 and 4.9.

17. A pharmaceutical composition or food supplement according to claim 14 comprising an Extract of natural Ginkgo biloba and an Extract of natural Picrorrhiza kurroa which are a synergistic combination of Extracts.

18. A pharmaceutical composition or food supplement according to claim 17 comprising combinations of the following Extracts as herein before described: Extracts 17 and 59; 10 and 41; 17 and 50; 17 and 41; and 17 and 54; and combinations or compositions substantially similar to these combinations.

19. A pharmaceutical composition or food supplement according to claim 14 comprising two (or more) Extracts of natural Ginkgo biloba, each of which Extracts, if isolated, gives a substantially positive result in a neutrophil oxidative burst assay and which represent a synergistic combination of Extracts.

20. A pharmaceutical composition or food supplement according to claim 19 comprising combinations of the following Extracts of Ginkgo biloba as hereinbefore described: Extract Numbers 50, 54 and 59; 54 and 59; 54 and 64; 42 and 44; 50, 59 and 64; 42, 43 and 44; 50 and 59; 54, 59 and 64; 50 and 54; 50 and 64; 43 and 44; 50, 54 and 64; 42, 43 and 44; 50 and 64 and combinations of Extracts or compositions substantially similar to any of these.

21. A pharmaceutical composition or food supplement according to claim 14 comprising two (or more) Extracts of natural Picrorrhiza kurroa, each of which Extracts, if isolated, gives a substantially positive result in a neutrophil oxidative burst assay and which together represent a synergistic combination of Extracts.

22. A pharmaceutical composition or food supplement according to claim 21 comprising combinations of the following Extracts of Picrorrhiza kurroa as hereinbefore described: Extracts 16 and 17; 17 and 33; 10 and 17; 30 and 33; 30 and 32; 32 and 33; 14 and 18; 17 and 18; and 17 and 54, and combinations of Extracts or compositions substantially similar to any of these combinations.

23. A pharmaceutical composition or food supplement according to claim 1 which gives an Assay score of 10% or greater in the neutrophil oxidative burst assay set out in section 3 herein.

24. A pharmaceutical composition or food supplement according to claim 1 which gives an Assay score of 50% or greater in the neutrophil oxidative burst assay set out in section 3 herein.

25. A pharmaceutical composition or food supplement according to claim 1 which further comprises an antioxidant which inhibits the activation of the NF-kB cascade and/or interferes with the arachidonic acid cascade.

26. A pharmaceutical composition according to claim 23 wherein the antioxidant which inhibits the activation of the NF-kB cascade and/or interferes with the arachidonic acid cascade is selected from apocynin or apocynin derivative or acetosyringenin.

27. A pharmaceutical composition or food supplement according to claim 1 obtained or obtainable by a method comprising the steps of:
a) reverse phase chromatography of natural Picrorrhiza kurroa; and
b) normal phase chromatography of the product of step (a) to remove (or lower the proportion of) component(s) of natural Picrorrhiza kurroa.

28. A pharmaceutical composition according to claim 27 wherein chromatography step (b) removes component(s) having log P of −2 or less or log P of greater than +5.

29. A pharmaceutical composition or food supplement according to claim 1 obtained or obtainable by a method which comprises a step of solvent extraction from the Ginkgo biloba or Picrorrhiza kurroa.

30. A pharmaceutical composition comprising
a mixture of apocynin or apocynin derivative and Ginkgo biloba which includes less of a component of natural Ginkgo biloba than the amount of that component present in natural Ginkgo biloba;
which Component, if isolated as a single species, gives a substantially negative result in a neutrophil oxidative burst assay.

31. A method of treatment of disease in a human or animal patient comprising a step of administration to the patient a pharmacologically active amount of a pharmaceutical composition according to claim 1.

32. A method according to claim 29 for the treatment or amelioration or relief of inflammatory disease.

33. The use of a mixture defined in claim 1 as a medicament, or in the manufacture of a medicament, for the treatment or amelioration or relief of inflammatory disease.
